(19) United States
(12) Patent Application Publication
(10) Pub. No.: US 2006/0099243 A1
(43) Pub. Date: May 11, 2006

(54) Liposome drug delivery

(75) Inventor: Milton B Yatvin, Portland, OR (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Oradel Medical Ltd.

(21) Appl. No.: 10/514,755

(22) PCT Filed: May 19, 2003
(86) PCT No.: PCT/US03/15789

§ 371 Date: Dec. 28, 2005

Related U.S. Application Data
(60) Provisional application No. 60/382,216, filed on May 20, 2002.

(51) Int. Cl. A61K 9/127; A61K 9/20; A61K 48/00; A61K 38/43

(52) U.S. Cl. 424/450; 424/464; 514/44; 424/94.1

(57) ABSTRACT

The invention comprises pharmaceutical compositions for administering a biologically active compound to an animal. Particularly provided are proliposomal compositions that are advantageously used to deliver biologically active compounds to the gastrointestinal tract after oral administration.

### BACKGROUND OF THE INVENTION

1. Field of the Invention

This invention relates to delivery of drugs, nutrients and other compounds to a biological organism. In particular, the invention relates to liposomes and formulations of drugs, nutrients and other compounds into liposomes to improve or effect delivery of such beneficial compounds to cells and tissues in an organism. Specifically, the invention provides such liposome compositions of drugs, nutrients and other compounds in formulations advantageously administered orally to an animal.

2. Background of the Related Art

A major goal in the pharmacological arts has been the development of reagents and methods that reduce the necessity of administering therapeutic compounds, drugs and other agents invasively (i.e., such as by injection). Most preferably, it has been a consistent goal in the art to develop therapeutic compounds, drugs and agents and formulations thereof that permit oral administration (see, for example U.S. Pat. No. 4,963,526 to Ecanow issued Oct. 16, 1990), although other reduced-invasiveness formulations such as suppositories have also been developed. Among the various routes of drug administration, the oral intake of drugs is undoubtedly preferred because of its versatility, safety and patient comfort.

In addition, it has been a goal in the nutritional arts to develop preparations that increase transit of certain nutrients through the gastrointestinal tract to increase uptake and delivery of such nutrients into the bloodstream. In particular, such preparations have been developed to permit chemically-labile nutrients (such as vitamins and other sensitive compounds) to pass through the chemically-hostile environment of the stomach for absorption in the intestines (see, for example, U.S. Pat. No. 5,958,450 to Tashiro issued Sep. 28, 1999). Preparations having enhanced intestinal uptake have also been deemed desirable.

One approach known in the prior art for improving efficiency of delivery of therapeutic compounds, drugs and other agents has been to envelop such compounds in a specialized lipid structure termed a liposome (see, for example, U.S. Pat. No. 4,744,989 to Payne et al. issued May 17, 1988). Liposomes generically comprise an enclosed lipid droplet having a core, typically an aqueous core, containing the compound. In certain embodiments, the compound is chemically conjugated to a lipid component of the liposome. In other embodiments, the compound is simply contained within the aqueous compartment inside the liposome.

Certain liposome formulations are known in the art U.S. Pat. No. 5,223,263, issued Jun. 29, 1993 to Hostetler et al. discloses conjugates between antiviral nucleoside analogues and polar lipids for inclusion in liposomes.

U.S. Pat. No. 5,466,468 to Schneider et al. issued Nov. 14, 1995 discloses parenterally administrable liposome formulations comprising synthetic lipids.

U.S. Pat. No. 5,484,809, issued Jan. 16, 1996 to Hostetler et al. discloses taxol and taxol derivatives conjugated to phospholipids.

U.S. Pat. No. 5,580,571, issued Dec. 3, 1996 to Hostetler et al. discloses nucleoside analogues conjugated to phospholipids.

U.S. Pat. No. 5,626,869 to Nyqvist et al. issued May 6, 1997 discloses pharmaceutical compositions wherein the pharmaceutically active compound is heparin or a fragment thereof contained in a defined lipid system comprising at least one amphiphatic and polar lipid component and at least one nonpolar lipid component.

U.S. Pat. No. 5,744,461, issued Apr. 28, 1998 to Hostetler et al. discloses nucleoside analogues conjugated to phosphonoacetic acid lipid derivatives.

U.S. Pat. No. 5,744,592, issued Apr. 28, 1998 to Hostetler et al. discloses nucleoside analogues conjugated to phospholipids.

U.S. Pat. No.5,756,116, issued May 26, 1998 to Hostetler et al. discloses nucleoside analogues conjugated to phospholipids.

U.S. Pat. No.5,843,509 to Calvo Salve et al. issued Dec. 1, 1998 discloses stabilization of colloidal systems through the formation of lipid-polysaccharide complexes comprising a water soluble and positively charged polysaccharide and a negatively charged phospholipid.

International Patent Application Publication Number WO89/02733, published April 1989 to Vical discloses conjugates between antiviral nucleoside analogues and polar lipids.

European Patent Application Publication Number 0350287A2 to Vical discloses conjugates between antiviral nucleoside analogues and polar lipids.

International Patent Application Publication Number WO93/00910 to Vical discloses conjugates between antiviral nucleoside analogues and polar lipids.

Rahman et al., 1982, Life Sci. 31: 2061-71 found that liposomes which contained galactolipid as part of the lipid appeared to have a higher affinity for parenchymal cells than liposomes which lacked galactolipid.

Gregoriadis, 1995, Trends in Biotechnology 13: 527-537 reviews the progress and problems associated with using liposomes for targeted drug delivery.

Ledley, 1995, Human Gene Therapy 6: 1129-1144 reviews the use of liposomes for gene therapy.

Mickisch, 1995, World J. Urology 13: 178-185 reviews the use of liposomes for gene therapy of renal cell carcinoma.

Yang et al. 1997, J. Neurotrauma 14: 281-297 review the use of cationic liposomes for gene therapy directed to the central nervous system.

Storm & Crommelin, 1997, Hybridoma 16: 119-125 review the preliminary use of liposomes for targeting chemotherapeutic drugs to tumor sites.

Manusama et al., 1998, Semin. Surg. Oncol. 14: 232-237 report on preclinical and clinical trials of liposome-encapsulated tumor necrosis factor for cancer treatments.

Although liposomes have conventionally been administered parenterally (see, for example, U.S. Pat. No.5,466,468), reports of oral administration of liposome-related formulations have appeared in the art.

U.S. Pat. No. 4,921,757 to Wheatley et al. issued May 1, 1990 discloses controlled release of biologically active substances, such as drugs and hormones entrapped in liposomes which are protected from the biological environment by encapsulation within semi-permeable microcapsules or a permeable polymeric matrix.

U.S. Pat. No. 5,043,165 to Radhkrishnan to Aug. 27, 1991 disclosed a liposome composition for sustained release of steroidal drugs.

U.S. Pat. No, 5,762,904 to Okada et al. issued Jun. 9, 1998 discloses oral delivery of vaccines using polymerized liposomes.

U.S. Pat. No. 5,955,451 to Lichtenberger et al. issued Sep. 21, 1999 discloses compositions comprising non-steroid anti-inflammatory drugs (NSAID's) complexed with either zwitterionic or neutral phosphollpids, or both, having reduced gastrointestinal irritating effects and enhanced antipyretic, analgesic, and anti-inflammatory activity.

Proliposomes are an alternative to conventional liposomal formulations. Proliposomes are dry, free-flowing granular products, which, on addition of water, disperse to form a multi-lamellar liposomal suspension. The stability problems associated with conventional liposomes such as aggregation, susceptibility to hydrolysis and/or oxidation are avoided by using proliposomes.

U.S. Pat. No. 5,635,206 to Ganter et al. issued Jun. 3, 1997 discloses a process for preparing liposomes or proliposomes.

Proliposomes of indomethacin were prepared using effervescent granules, which upon hydration yielded liposomes of high encapsulation efficiency and increased anti-inflammatory activity with decreased ulcerogenic index (see, for example, Katare et al., 1991, J. Microencapsulation 81: 1-7).

The proliposomal concept has been extended to administer drugs through various routes and also to the food industry wherein enzyme immobilization is essential for various food processing regimes. A typical example is the immobilization of the enzyme, chymotrypsin, in liposomes obtained from proliposomes.

There remains a need in the art for a general, inexpensive and effective means for delivering biologically-active compounds, including drugs, hormones, enzymes, genetic material, antigens including vaccines, and nutrients, to an animal by oral administration. Advantageous embodiments of such delivery means are formulated to efficiently deliver biologically-active compounds to the appropriate portion of the gastrointestinal tract for efficient absorption.

### SUMMARY OF THE INVENTION

The present invention is directed to an improved method for delivering biologically-active compounds, particularly drugs, hormones, enzymes, genetic material, antigens including vaccines, and nutrients, to an animal by oral administration. This delivery system achieves specific delivery of such biologically-active compounds through associating the compounds with liposomes and proliposome components.

In preferred embodiments, the biologically active compound is formulated as a proliposomal composition that can be reconstituted in vivo to provide a liposomal preparation. Preferably, the invention provides pharmaceutical compositions comprising the biologically active compound and a lipid formulated as a proliposomal preparation. In more preferred embodiments, the pharmaceutical compositions of the invention are formulated for oral administration. Most preferably, the pharmaceutical compositions of the invention formulated for oral administration comprise an enteric coating sufficient to prevent dissolution of the composition in the stomach of an animal. In alternative embodiments, the pharmaceutical compositions also comprise a protective coating between the enteric coating and the core of the composition comprising the proliposomal components thereof. Additional advantageous components of said orally-administrable pharmaceutical compositions further comprise the pharmaceutical compositions as will be understood by those with skill in the art.

In preferred embodiments, the pharmaceutical compositions of the invention further comprise a chitosan layer between the proliposomes and the enteric coating.

Specific preferred embodiments of the present invention will become evident from the following more detailed description of certain preferred embodiments and the claims.

### BRIEF DESCRIPTION OF THE DRAWINGS

FIGS. 1A through 1C depict thermograms produced by differential scanning calorimetry as set forth in Example 1.

FIGS. 2 and 3 depict transfer rates of glyburide through a Caco-2 cellular monolayer using the liposomal compositions of the invention, as set forth in Example 2.

FIGS. 4 and 5 depict total accumulation of glyburide in the receiving chamber of a transwell comprising a Caco-2 cellular monolayer using the liposomal compositions of the invention, as set forth in Example 2.

### DETAILED DESCRIPTION OF THE PREFERRED EMBODIMENTS

The present invention provides compositions of matter and methods for facilitating the delivery of biologically-active compounds to the tissues of an animal after oral administration. For the purposes of this invention, the term “biologically-active compound” is intended to encompass all naturally-occurring or synthetic compounds capable of eliciting a biological response or having an effect, either beneficial or deleterious, including cytotoxic, on biological systems, particularly tissues, cells and cellular organelles. These compounds are intended to include but are not limited to all varieties of drugs, including but not limited to antibiotic, antibacterial, antiviral, antimycotic, anti-inflammatory, antiproliferative and antineoplastic drugs; hormones, including peptide hormones and steroid hormones, and most particularly including endocrine and exocrine gland hormones; genes, recombinant nucleic acids, oligonucleotides or other nucleic acids encoding all or a portion of a mammalian gene, a viral gene or a gene from a microorganism; antigens, particularly in the form of vaccines; enzymes, particularly digestive enzymes and most particularly enzymes involved in processing, modifying, converting or degrading a nutrient into a form more easily absorbed by an animal's gastrointestinal tract; nutrients, and most preferably vitamins and minerals; and most particularly any biologically active compound, including particularly nutrients, that inefficiently transits the gastrointestinal tract or is unstable in a compartment thereof.

Pharmaceutical compositions comprising the biologically active compounds of the invention are preferably provided as proliposomal compositions that can be reconstituted, most preferably in vivo, to produce liposomal compositions of the biologically active compounds. As used herein, the term “proliposome” and “proliposomal” are intended to encompass dry, free-flowing granular products, which, on addition of water, disperse to form multi-lamellar liposomal suspensions comprising the biologically active compounds of the invention. Advantageously, the stability problems associated with the conventional liposomes (such as aggregation, susceptibility to hydrolysis and oxidation) are avoided by using proliposomes

The proliposomal compositions provided by the invention are reconstituted, particularly in vivo, to provide liposomal compositions wherein the biologically active compounds of the invention are encapsulated in said liposomes. In preparing the proliposomal compositions of the invention, lipid components including neutral lipids, positively-charged lipids, negatively-charged lipids, amphoteric lipids such as phospholipids, and cholesterol are advantageously used. As defined herein, the “lipid component” of the proliposomal compositions of the invention are intended to encompass a single species of lipid (such as a particular phospholipid) or combinations of such lipids, either of one type such as combinations of phospholipids (for example, phosphatidylcholine plus phosphatidyl ethanolamine) or of different types (such as a phospholipid plus a charged lipid or a neutral lipid). Combinations comprising a multiplicity of different lipid types are also advantageously encompassed by the proliposomal compositions of the invention (see, Lehninger, 1975, Biochemistry, 2d ed., Chapters 11 & 24, Worth Publishers: New York; and Small, 1986, “From alkanes to phospholipids,” Handbook of Lipid Research: Physical Chemistry of Lipids, Volume 4, Chapters 4 and 12, Plenum Press: New York).

Biologically active compounds that are unstable in the stomach, such as proteins and peptides, vitamins and other small molecule nutrients, or biologically active compounds that irritate the stomach, such as various analgesics like aspirin and other non-steroidal anti-inflammatory drugs (NSAIDs), and those compounds that are preferentially absorbed in the small intestine are preferred biological compounds useful with the liposomal formulations of the invention. In preferred embodiments, said compounds include but are not limited to aspirin, ibuprofen, erythromycin, vasopressin, insulin, dideoxyinosine (ddI), cyclosporine, taxol, heparin, halofantrine, ethopropazine, griseofulvin, propofol, furosemide, carbamazepine, diazepam, candesartan and cilexetil.

The proliposomal preparations comprising the biologically active compounds of the invention are preferably provided in a form that can be orally administered, including but not limited to syrups, elixirs, capsules, tablets, and emulsions. Preferred forms are tablets or capsules, most preferably comprising an enteric coating to prevent premature dissolution under the chemically harsh environment of the stomach Enteric coatings are prepared as will be understood by one having skill in the art, and preferably include coatings including but not limited to eudragit and cellulose acetate phthalate.

In alternative embodiments, the tablets or capsules of the invention comprise a protective coating between the enteric coating and the core of the capsule or tablet comprising the proliposomal preparations of the invention. In such embodiments, the protective coating is prepared as will be understood by one having skill in the art, and preferably include coatings including but not limited to hydroxypropyl methylcellulose, polyethylene glycol and ethylcellulose. In additional embodiments, the protective coating further comprises a plasticizing agent, including but not limited to triethylcitrate and polyvinyl pyrrolidone.

The tablets, capsules and other like embodiments of the proliposomal preparations and pharmaceutical compositions of the invention further advantageously comprise particle lubricants that minimize the tendency of the granular proliposomal compositions to agglomerate. By “article lubricant” as used herein is meant the class of materials used in the manufacturing of pharmaceutical tablets as lubricants to improve the flowability and prevent agglomeration of an active agent during the tableting process. Examples of particle lubricants include talc, lactose, corn starch, ethyl cellulose, fatty acid salts such as magnesium stearate, agar pectin, fatty acids such as stearic acid, gelatin and acacia.

The invention in alternative embodiments comprises a chitosan layer between the proliposomes and the enteric coating. As used herein, the term “chitosan” is used to specifically refer to poly(1,4-β-D-glycopyrano-amine), and to substituted derivatives and analogues thereof having substantially the same properties as the base polymer. Preferably, the chitosan of the invention is hydrolyzed, deacetylated chitin obtained from, inter alia, shrimp, crab or other crustacean shells. As used herein, the chitosan layer can be produced on the proliposome formulations of the invention by phase separation of chitosan from an aqueous solution by a counterion (termed simple coacervation; see Chandy and Sharma, 1992, Biomaterials 13: 949-952). Alternatively, the chitosan layer can be produced on the proliposome formulations of the invention by phase separation of chitosan from an aqueous solution by an oppositely-charged macromolecule (termed complex coacervation; see Polk et al., 1994, Aquacultural Engineer. 13: 311-323). In another embodiment, the chitosan layer can be produced on the proliposome formulations of the invention by emulsification of the chitosan and proliposomes in a non-solvent (see Akbuga and Durnaz, 1994, J. Intl. Pharmaceutics 111: 217-222; Jameela and Jayakrisnan, 1995, Biomaterials 16: 769-775). Chitosan preparations having varying degrees of deacetylation or average molecular weight are useful in the practice of the invention (see, Chiuo et al., 2001, J. Microencapsul. 18: 613-625).

The invention specifically provides methods for preparing and administering the proliposomal compositions of the invention as disclosed in the Examples below, and pharmaceutical compositions comprising the proliposomal preparations of biologically active compounds.

Animals to be treated with the proliposomal preparations and pharmaceutical compositions of the invention are intended to include all vertebrate animals, preferably domesticated animals, such as cattle, horses, goats, sheep, fowl, fish, household pets, and others, as well as wild animals, and most preferably humans.

One advantage of orally-administered liposomal formulations over parenterally-administered formulations is that oral administration reduces uptake of liposomes by the liver, thus reducing liver toxicity (which is a particular liability of parenterally-administered liposomal formulations). Oral formulations are targeted to deliver biologically active compounds to the intestine, which is a large surface for absorption and results in slow release of the administered compound. Finally, oral administration avoids transport-mediated saturation of drugs like dideoxyinosine.

The formulations of the invention are also advantageously used for treating diseases that cause or result in malabsorption, including but not limited to Crohn's disease, irritable bowel syndrome, celiac sprue, diverticulitis, immunoproliferative small intestine disease, liver disease, diseases and disorders of the gall bladder (including those disorders that are consequent to'surgical removal of the gall bladder), pancreatitis, Schwachman's syndrome, steatorrhea, Whipple's disease, parasitic infection, malabsorption as a consequence of chronic laxative use or abuse, pancreatic enzyme deficiency, disaccharidase deficiency, or defects in fat absorption consequent to surgical gastrectomy or other surgical interventions in the gastrointestinal tract.

The following Examples illustrate certain aspects of the above-described method and advantageous results. The following examples are shown by way of illustration and not by way of limitation.

### EXAMPLE 1

Proliposomal formulations useful for oral administration were developed using an in vitro model system. Human Caco-2 cells (colon adenocarcinoma cells), grown on semipermeable filters, provide a simple and reliable in vitro model for studying drug transport across the intestinal mucosa. Caco-2 cells are recognized in the art for yielding useful predictions on oral absorption of new drug formulations.

1. Preparation of Proliposomal Formulations

In order to assay the proliposomal tablets of the invention, glyburide (glybenclamide), an oral blood-glucose-lowering drug of the sulfonylurea class, was used as model drug, because uptake in the CaCo-2 system can be monitored by measuring transport across monolayers formed by this cell line.

Proliposomal tablets were prepared as follows. The identities and amounts of each of the reagents used to prepare the tablets of the invention are shown in Table I. Phospholipids DMPC and DSPC were obtained from Avanti Polar Lipids (Alabaster, Ala.); glyburide, cholesterol, stearylamine, dicetylphosphate and all tissue culture reagents, were obtained from Sigma Chemical Co. (St. Louis, Mo.); purified talc and anhydrous lactose were obtained from J.T. Baker (Phillipsburg, N.J.) and Quest, Int'l. (Hoffman Estates, Ill.); chloroform, methanol and ethanol were obtained from Fisher Scientific (Fairlawn, N.J.); Caco-2 cells were obtained from the American Type Culture Collection (Manassas, Va.; Accession No. HTB 37); and transwell culture chambers were obtained from Costar (Cambridge, Mass.).

Glyburide, lipid and cholesterol were dissolved at room temperature in 10 mL chloroform. Lactose (25 mg/tablet) was suspended in the organic mixture and the suspension evaporated to dryness at 60° C. in a conventional coating pan (pan drying method). The solid residue was collected and sifted through a #60 mesh screen. The sifted residue was then mixed with Explotab® (3 mg/tablet), lactose (50 mg/tablet) and talc (2 mg/tablet) and compressed into tablets using a Manesty B3B 16 station press. The tablets were then coated with a solution of hydroxypropyl methylcellulose in ethyl alcohol (3% w/v) containing triethyl citrate (15% of polymer weight) as a plasticizer. Eudragit L30 D-55 (7% w/w) was then applied on the coated tablets.

Table I provides a formulary for preparing proliposomal tablets according to the invention.

In alternative methods, proliposomal formulations can be prepared by lyophilization. In these embodiments, mixtures of lipids and drug are prepared in aqueous solution and then sonicated, causing small unilamellar liposomes to form and resulting in an optically-clear solution. Such a solution is then freeze-dried and mixed with the other components of the tablets as described above. This method has the advantages that it can be performed in five steps, and avoids the use of organic solvents, which can be toxic, in preparing the formulation.

TABLE I
Formulary for Preparing Proliposome Tablets

| | Quantity of each ingredient used (mg/tablet) | | | | | |
|---|---|---|---|---|---|---|
| Formulation | Glyburide | DSPC | DMPC | CHO | STA | DCP |
| DSPC/Neu | 5.0 | 10.0 | — | — | — | — |
| DSPC.Neu.Cho | 5.0 | 10.0 | — | 2.45 | — | — |
| DSPC/Pos | 5.0 | 10.0 | — | — | 0.35 | — |
| DSPC/Pos/Cho | 5.0 | 10.0 | — | 2.45 | 0.35 | — |
| DSPC/Neg | 5.0 | 10.0 | — | — | — | 0.69 |
| DSPC/Neg/Cho | 5.0 | 10.0 | — | 2.45 | — | 0.69 |
| DMPC/Neu | 5.0 | — | 10.0 | — | — | — |
| DMPC/Neu/Cho | 5.0 | — | 10.0 | 2.85 | — | — |
| DMPC/Pos/Cho | 5.0 | — | 10.0 | 2.85 | 0.40 | — |

DSPC = distearylphosphatidylcholine
DMPC = dimyristylphosphatidylcholine
STA = stearylamine (Pos: positively charged lipid)
CHO = cholesterol (Neu: neutral lipid)
DCP = dicetylphosphate (Neg: negatively charged lipid)

In other alternative methods, proliposomal formulations can be prepared by spray-drying. In these embodiments, mixtures of lipids and drug are prepared in aqueous solution. To such a mixture is added a surfactant such as Tween 80®, and then dried using a spray dryer. The resulting dried proliposomal preparation is mixed with the other components of the tablets as described above. This method has the advantages that it can be performed in five steps, is suitable for use with temperature-sensitive materials, and avoids the use of organic solvents, which can be toxic, in preparing the formulation.

In another embodiment of this alternative method, a mechanical mixer is used instead of using a surfactant. The mechanical mixer produces a proliposomal composition in the absence of a surfactant that can be spray-dried as described above. This embodiment is particularly advantageous because it avoids the use of both surfactants and organic solvents in preparing proliposomal formulations according to the invention.

2. Chemical Assays of Reagents and Proliposomal Formulations

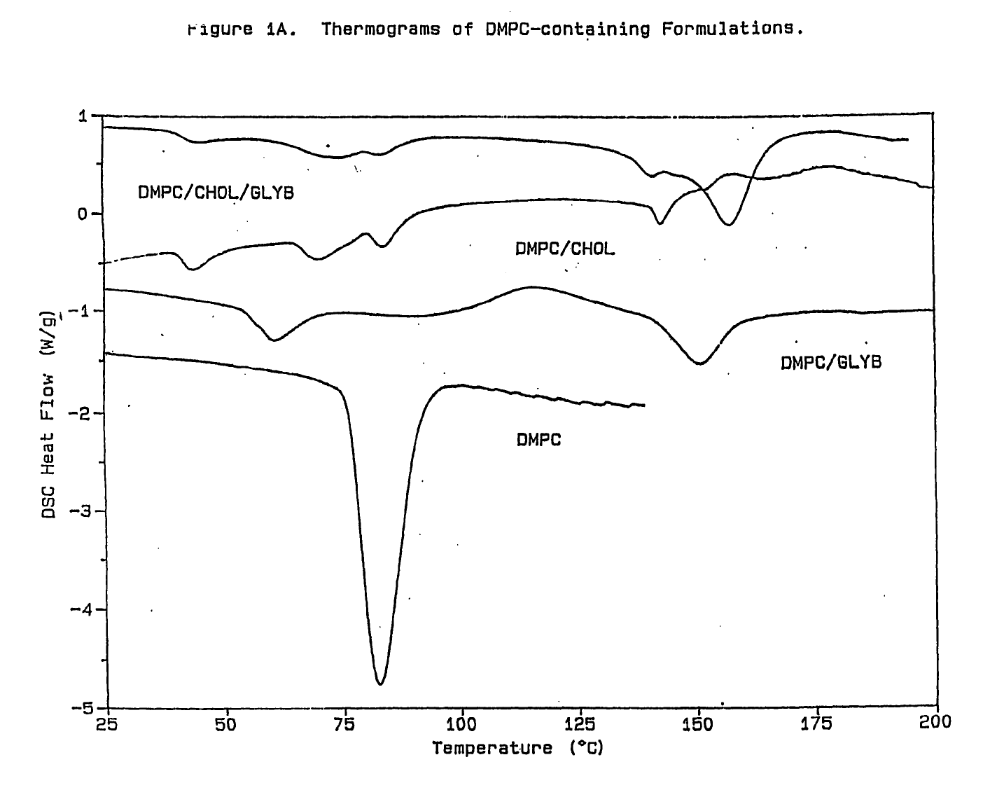

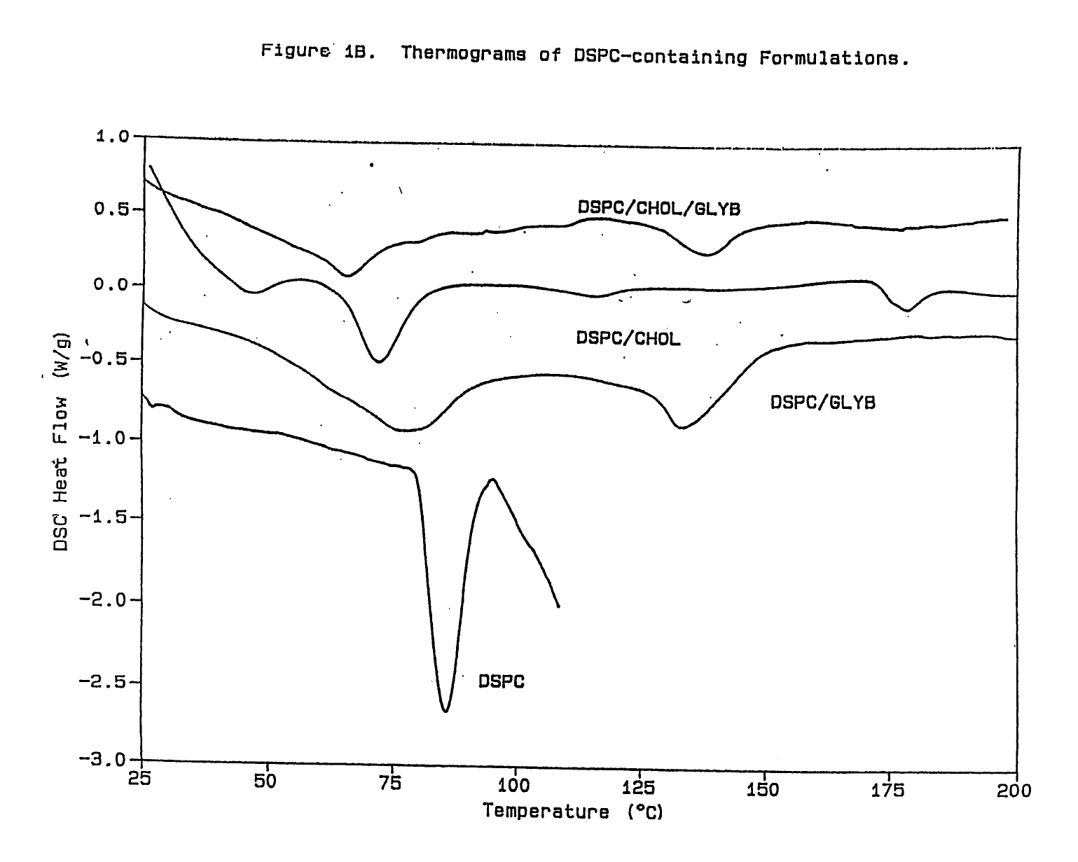

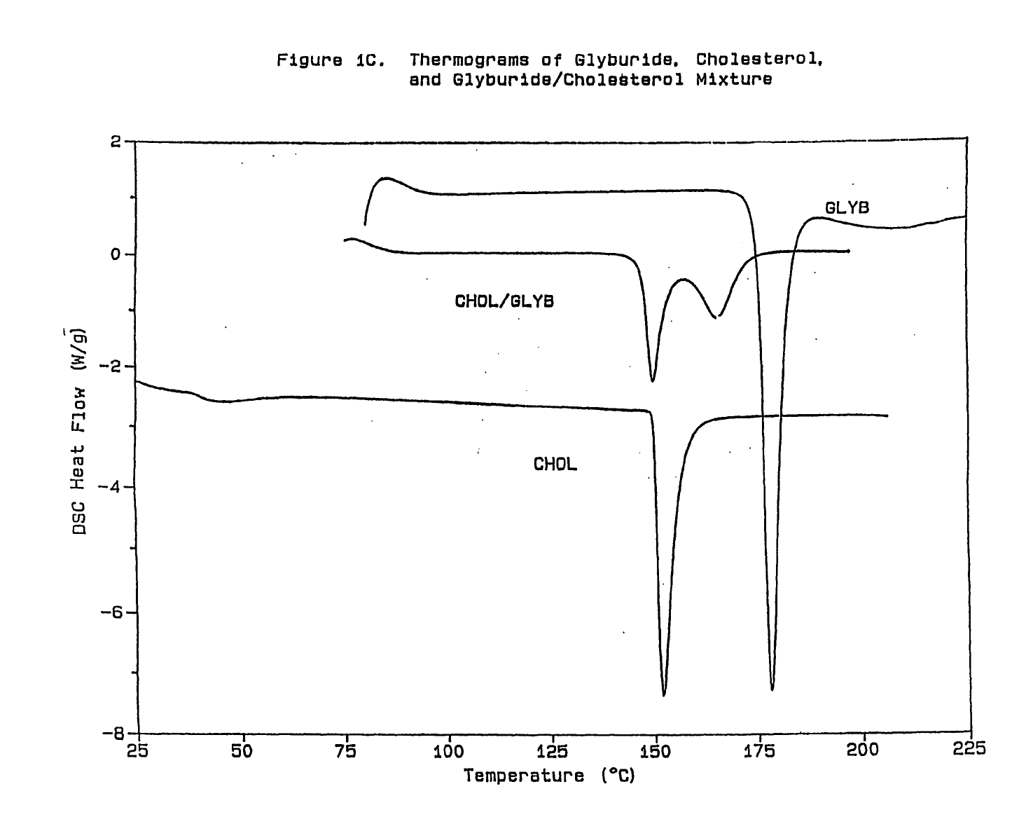

The purity of the reagents used to make the proliposome tablets of the invention described herein was tested using differential scanning calorimetry. Samples were prepared by dissolving lipid with glyburide and cholesterol separately at a ratio of 1:1 (w/w) in an excess of chloroform. The organic layer was removed and thermograms obtained using a differential scanning calorimeter (TA Instruments, New Castle, Del., Model 2910). Each component was scanned both individually and using a mixture comprising glyburide, lipid and cholesterol at a ratio of 1:1:1 (w:w:w). 2-5 mg of sample was scanned at a rate of 20° C. per minute over a suitable temperature range (25-225° C.) in a hermetically-sealed aluminum pan. The peak transition temperatures of the dispersion were compared with the pure compounds. The results of these experiments are shown in FIGS. 1A through 1C.

FIG. 1A shows a thermogram of DMPC alone compared with mixtures of DMPC and cholesterol (DMPC/CHOL), DMPC and glyburide (DMPC/GLYB) and DMPC, cholesterol and glyburide (DMPC/CHOL/GLYB). Peak transition temperatures are shown in the Figure. In contrast to the simple and easily-recognizable peak transition temperature obtained for DMPC, the mixtures are heterogeneous, having more than one localized peak region where a thermal transition occurs.

FIG. 1B shows a thermogram of DSPC alone compared with mixtures of DSPC and cholesterol (DSPC/CHOL), DSPC and glyburide (DMPC/GLYB) and DSPC, cholesterol and glyburide (DSPC/CHOL/GLYB). Peak transition temperatures are shown in the Figure. A similar pattern is observed herein, where there is a simple and easily-recognizable peak transition temperature obtained for DSPC, but the mixtures are heterogeneous, having more than one localized peak region where a thermal transition occurs.

Thermograms were also obtained individually and in mixtures for glyburide and cholesterol, and these results are shown in FIG. 1C. From these thermograms, it is evident that the presence of cholesterol acts as an “impurity” in the drug, lowering its melting point. The same effect is observed in mixtures of the drug and lipid. In the presence of both cholesterol and lipid, the melting point of glyburide is further decreased, demonstrating a synergistic effect. These results also indicate that the amount of heat required to melt the drug in a pure state is far higher than the amount needed when the drug is combined with cholesterol or lipid. This explains the increased solubility of the drug when prepared in a solid dispersion of lipid and/or cholesterol.

Liposomes were reconstituted from proliposomal tablets by adding one tablet to 1 mL phosphate buffered saline in a sterile glass vial. The tablet was allowed to stand at 37° C. for 1 hour with shaking, which was sufficient to dissolve the tablet and reconstitute the liposomal preparation.

Reconstituted liposomes were characterized for size distribution by large-angle dynamic light scattering using a particle size analyzer (Brookhaven Instruments, Model BI-90). Each preparation was diluted with filtered saline to an appropriate concentration to achieve a medium viscosity of 0.089 centipoise and a medium relative refractive index of 1.332 at room temperature. Measurements obtained under these condition are shown in Table II. These results indicated that the particle size of the resulting liposomes varied both with the presence or absence of cholesterol and with the identity of the phospholipid component The mean diameter of the liposomes was greater in neutral liposome embodiments than in charged liposome embodiments, and can be explained by the greater propensity of neutral liposomes to aggregate or fuse with one another.

TABLE II
Liposome Particle Size (nm) of Different Tablet Formulations

Lipid Type

| Formulation/Charge | DSPC | DMPC |
|---|---|---|
| Neutral | 1413 | 1825 |
| Neutral/Cholesterol | 1035 | 748 |
| Positive | 1059 | N.D. |
| Positive/Cholesterol | 867 | 629 |
| Negative | 3633 | N.D. |
| Negative/Cholesterol | 800 | N.D. |

N.D.: not determined

Encapsulation efficiency, defined as the percentage of the glyburide encapsulated in liposomes, was determined using the protamine-induced aggregation method as described in Kulkarni et al. (1995, Pharm. Sci. 1: 359-362). Briefly, each tablet was disintegrated in 1 mL of phosphate-buffered saline (PBS, pH 7.4) to give a concentration of 10 mg/mL of lipid. To 100 μL of the preparation, equal quantities of a protamine solution (50 mg/mL) in PBS was added and vortexed for about 1 min. The mixture was then incubated for about 12 hours at room temperature. After incubation, the mixture was centrifuged at about 16,000×g for about 5 minutes. 100 μL of the supernatant was removed and the pellet was dissolved in about 1 mL of reagent-grade alcohol (95% ethanol) and sonicated for 5 minutes.

The quantity of glyburide in the pellet and the supernatant was determined by HPLC analysis using the Star® 9010 solvent system and Star 9095® variable-wavelength ultraviolet/visible spectrum spectrophotometric detector (Varian Associates, Walnut Creek, Calif.) and the data analyzed by a Dynamax® MacIntegrator (Rainin Instrument Co., Woburn, Mass.). HPLC analysis was performed using a C18 column (Phenominex®) packed with 5 μm particles and having dimensions of 250 mm in length and an internal diameter of 4.6 mm. The mobile phase was a solution of methanol in 0.1 M phosphate buffer, pH 3.5 at a ratio of 75:25 by volume. Column flow rate was 1.0 mL/min and the output was scanned at a wavelength of 225 nm.

The results of these characterization experiments are shown in Table III.

These results demonstrated that a slightly higher percentage of the drug was encapsulated in DMPC. These results are consistent with a slightly higher amount of the drug being encapsulated in “fluid” liposomes (i.e., those comprising DMPC) than liposomes in a gel state (i.e., those comprising DSPC) at 37° C.

TABLE III
Drug Encapsulation Efficiency (% ± s.d.)

Lipid Type

| Formulation | DSPC | DMPC |
|---|---|---|
| Neutral | 81.6 ± 0.4 | 86.7 ± 2.7 |
| Neutral/Cholesterol | 80.4 ± 0.6 | 88.8 ± 1.2 |
| Positive | 78.4 ± 0.7 | N.D. |
| Positive/Cholesterol | 81.0 ± 1.2 | 87.6 ± 0.6 |
| Negative | 81.2 ± 0.1 | N.D. |
| Negative/Cholesterol | 80.4 ± 0.4 | N.D. |

N.D.: Not determined

### EXAMPLE 2

Caco-2 cell cultures were prepared as monolayers on polycarbonate transwells having a membrane pore size of 4 nm. Caco-2 cells were first grown in T-150 flasks (Falcon, Lincoln Park, N.J.) at 37° C. under an atmosphere of 5% CO₂ and 95% air in Dulbecco's modified Eagle's medium (pH 7.2, Sigma Chemical Co., St Louis, Mo.), with conventional supplements. The medium was changed every other day until the monolayers reached about 90% confluency. Media was removed and the cells were washed with Hank's balanced salt solution (HBSS, Sigma). The cells were trypsinized by adding 0.5 mL of a 0.25% trypsin solution containing 1 mM EDTA to each flask and incubating the monolayers for 10 min at 37° C. The separated cells were removed from the flasks and collected into centrifuge tubes, centrifuged at 200×g for 10 min, the supernatant removed and the pellet resuspended in a sufficient amount of Dulbecco's modified Eagle medium to yield a suspension that would produce about 60,000 cells/cm² on plating. The Caco-2 cells were then seeded into Transwell semipermeable membrane inserts having 4 μm pore size. In the transwells, media was changed every other day until the cells were used for the transport studies described below.

Caco-2 cell cultures on transwell membranes prepared as described above were used for transport studies about 17 days after plating. Proliposome tablets were dissolved as described above by incubation for 1 h with shaking at 37° C. in 2 mL HBSS. As a control, pure glyburide treated with chloroform was compressed into tablet form with lactose and Explotab®; all controls were treated exactly as experimental.

The medium from the transwell plates was gently removed using a micropipette. 0.5 mL of the reconstituted liposomal suspension was gently added to the donor compartment of the transwell and 1.5 mL of HBSS was added to the receiver compartment 100 μL of FITC-Dextran was then added to the donor compartment to a final concentration of 10 μg/mL of FITC-Dextran in the donor side. FITC-Dextran was used as a marker to test for the presence of leaks, if any, on the monolayers covering the semipermeable transwell membranes. Samples (300 μL) were carefully withdrawn from the receiver side at 50, 120, 180, 240, 300 minutes after addition, and the receiver side was replenished with 300 μL of fresh HBSS each time the sample was taken. Cells were incubated at 37° C. in a 5% CO₂/95% air atmosphere at all times during these assays. Sampling was done under aseptic conditions in a laminar air-flow hood.

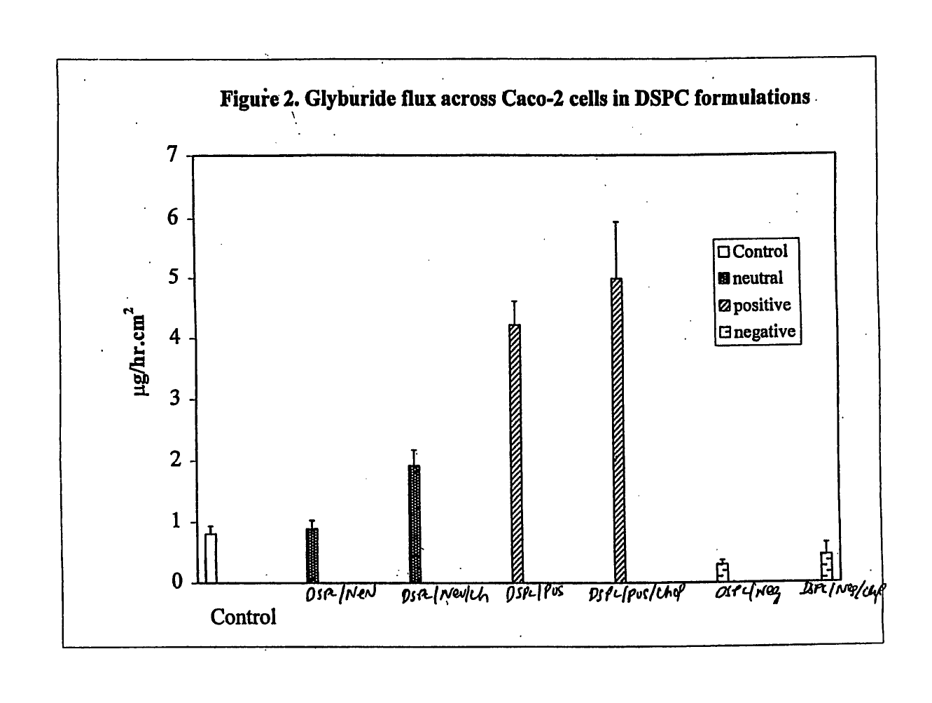

The amount of glyburide transported during each sampling interval was determined by injecting 90 μL of the sample onto the HPLC system described above in Example 1 and peak areas were recorded. These experiments were performed in triplicate and the average of the results was reported. The results of the experiments are shown in FIGS. 2 through 5.

FIG. 2 shows the results of glyburide transit across Caco-2 cell monolayers in formulations containing distearylphosphatidylcholine (DSPC). Control experiments performed in the absence of DSPC had a flow rate of almost 1 μg/hr·cm². Formulations of glyburide with DSPC (a “neutral” lipid at physiological pH) showed a similar level of flux across the monolayer, although the addition of cholesterol to these formulations increased the flux about two-fold. Formulations of glyburide with negatively-charged lipid, on the other hand, in either the presence or absence of cholesterol were transported across the monolayer at a lower rate. In contrast, formulations of glyburide with positively-charged lipid were transported across the membranes at a rate about fourfold higher than control, and the addition of cholesterol increased this to a rate of about fivefold higher than control.

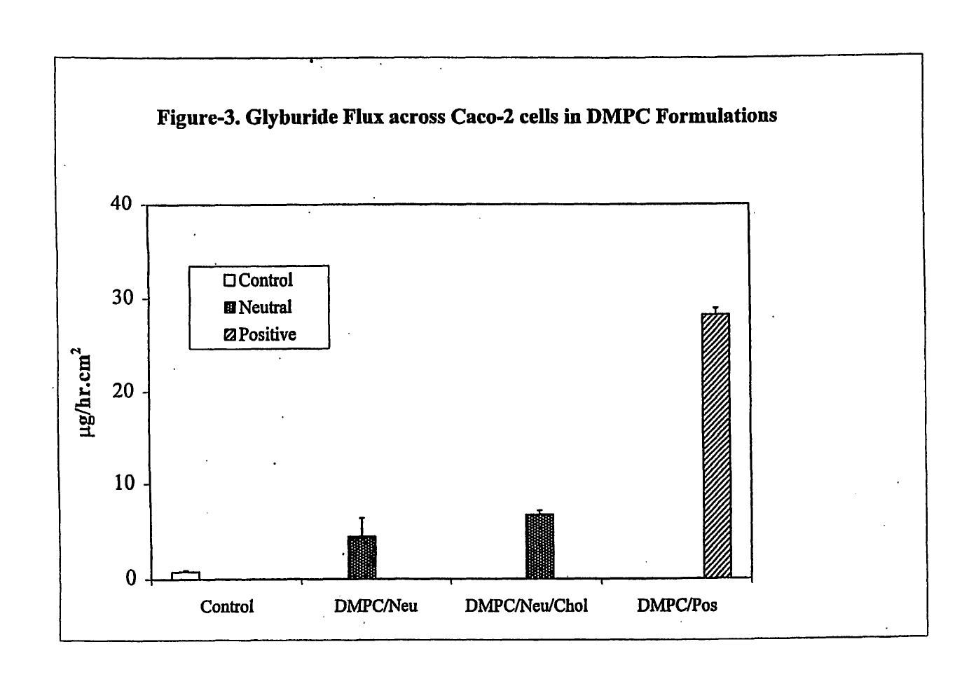

FIG. 3 shows the results of parallel experiments using dimyristylphosphatidylcholine (DMPC) as the lipid component. A similar pattern of glyburide flux was seen in these experiments; however, the degree of enhancement of transit across the Caco-2 cell monolayer was much higher for formulations containing DMPC. For example, glyburide formulations containing DMPC and positively-charged lipid had a transit rate almost thirty-fold higher than control. Formulations of neutral lipid were elevated to a lesser degree; in the presence of cholesterol such formulations had a transit rate about eightfold higher than control, and in the absence of cholesterol this rate was about fivefold higher than control.

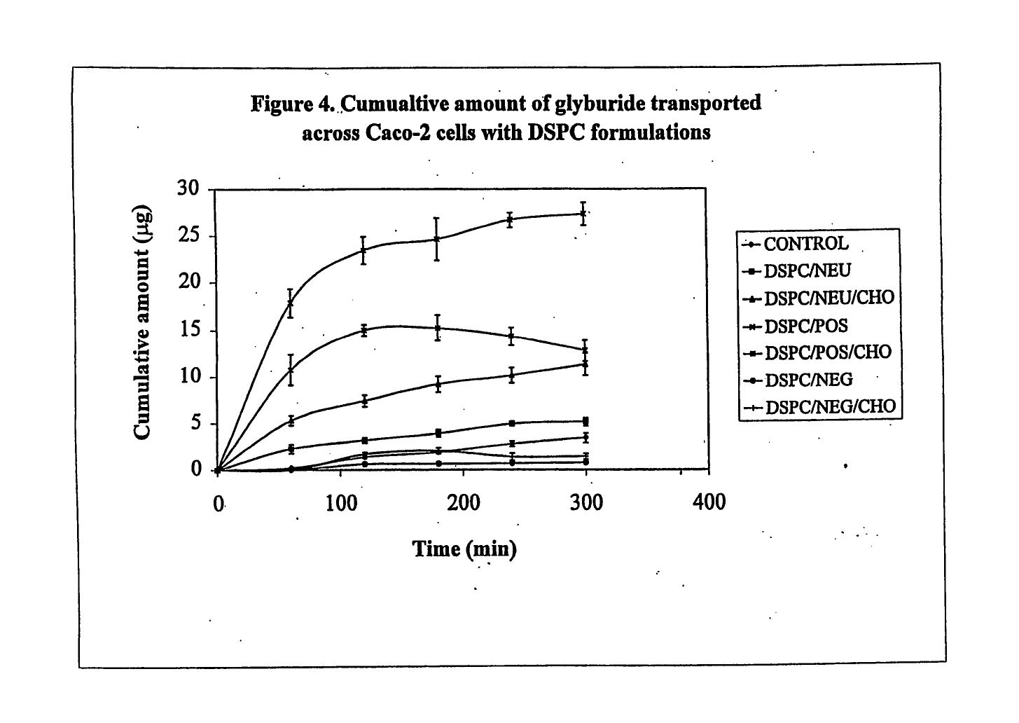

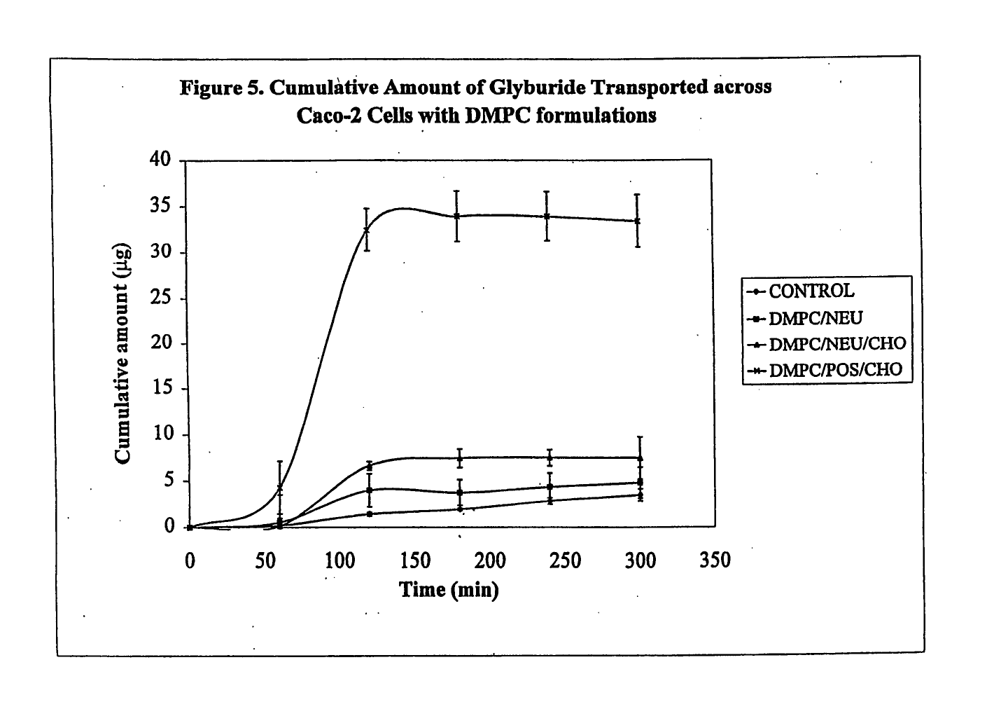

FIGS. 4 and 5 show the cumulative amount of transported glyburide using DSPC- and DMPC-containing formulations over a five hour period. FIG. 4 shows DSPC-containing formulations, wherein the highest accumulation levels were achieve with glyburide formulations containing DSPC and positively-charged lipid (about 27 μg). Similar formulations additionally containing cholesterol had lower total amounts (about 13 μg). DSPC formulations containing neutral lipid and cholesterol showed slower kinetics but achieved essentially the same total accumulation as DSPC/positive lipid/cholesterol formulations. Formulations containing DSPC and neutral lipids in the absence of cholesterol showed the same total accumulation as control (about 2.5 μg), while DSPC formulations with negatively-charged lipid (in the presence or absence of cholesterol) showed lower total accumulation amounts.

FIG. 5 shows the results of similar experiments performed with DMPC formulations. Total accumulation levels were noticeably higher than control only for formulations containing DMPC, positively-charged lipid and cholesterol (about 34 μg), while DMPC formulations with neutral lipid (in the presence or absence of cholesterol) resulted in total accumulation at levels equivalent to control (about 2-5 μg).

These results demonstrated that liposomes can be successfully prepared for oral administration in the form of enteric-coated proliposome tablets. The presence of cholesterol reduces the particle size of the formulation. Proliposomes provide a stable system of production of liposomes for oral administration. Degradation of proliposome contents of the tablet in the stomach can be effectively avoided by administering the proliposomes as enteric-coated tablets. Enhanced transport of glyburide across Caco-2 cells was observed with such liposomal formulations. Although the transport of glyburide with DMPC formulations is higher than transport in the DSPC formulation in vitro, DSPC formulations are better suited for in vivo conditions because of the rigidity and increased stability of the membrane against the attack of bile salts and enzymes of the intestine. Since in vitro transport across Caco-2 cells is an indication of bioavailability, an increased transport with the liposome formulation suggests an increased bioavailabilty of compounds that are poorly absorbed otherwise. For example, using a suitable polymer coating for the proliposomal tablets of the invention, colonic delivery of drugs, especially peptides may be possible.

Proliposomes are ideally suited for lipophilic compounds, since the majority of such a biologically active compound will partition into the lipid phase. These results also have implications for developing formulations that stabilize the encapsulated drug.

### EXAMPLE 3

### Producing a Chitosan Layer

A chitosan layer is produced on the proliposomal formulations of the invention as follows. Chitin is prepared according to conventional methods, for example, as disclosed in Chen et al. (1994, Carbohydrate Polym. 24: 41-46), and chitosan is prepared from chitin, for example, as disclosed in Yamamoto and Amaike (1997, Macromolecules 30: 3936-3937). Proliposomes are prepared as. described in Example 1 above. A 3% chitosan solution is prepared in 1% acetic acid solution, and about 2 mg of the proliposomes of the invention are poured into the chitosan solution and stirred until completely mixed. Spherical gels are produced from this mixture by adding a solution of 1N sodium hydroxide/26% ethyl alcohol using a small-bore (about 27 Ga) syringe and incubated for about 30 min. The chitosan-encased proliposomes are removed and rinsed in distilled water under neutral conditions and then dried for at least 24 h at room temperature.

It should be understood that the foregoing disclosure emphasizes certain specific embodiments of the invention and that all modifications or alternatives equivalent thereto are within the spirit and scope of the invention as set forth in the appended claims.

What is claimed is:

1. A pharmaceutical composition comprising a proliposomal preparation of a biologically active compound in a tablet comprising an enteric coating.

2. The pharmaceutical composition of claim 1 wherein the enteric coating is selected from the group consisting of eudragit and cellulose acetate phthalate.

3. The pharmaceutical composition of claim 1 further comprising a protective coating between the proliposomal preparation and the enteric coating.

4. The pharmaceutical composition of claim 3 wherein the protective coating is selected from the group consisting of chitosan, hydroxypropyl methylcellulose and polyethylene glycol.

5. The pharmaceutical composition of claim 3 wherein the protective coating further comprises a plasticizer.

6. The pharmaceutical composition of claim 5 wherein the plasticizer is selected from the group consisting of triethylcitrate and polyvinyl pyrrolidone.

7. The pharmaceutical composition of claim 1 wherein the biologically active compound is a nutrient, a hormone, a nucleic acid, an antibiotic drug, an enzyme, an antigen, an antiviral drug, an antiproliferative drug, an antineoplastic drug, an anti-inflammatory drug, a peptide or a protein.

8. The pharmaceutical composition of claim 1 wherein the lipid is a neutral lipid, a positively-charged lipid, a negatively charged lipid, a phospholipid or cholesterol, or combinations thereof.

9. The pharmaceutical composition of claim 8 wherein the phospholipid is phosphatidyl choline, phosphatidyl glycerol, phosphatidyl ethanolamine, phosphatidyl inositol, phosphatidyl serine, or phosphatidic acid.

10. The pharmaceutical composition of claim 8 wherein the positively-charged lipid is sphingosine, ceramide, stearylamine, or a cationic lipid.

11. The pharmaceutical composition of claim 8 wherein the negatively-charged lipid is dicetyl phosphate, phosphatidylserine or phosphatidyl glycerol.

12. The pharmaceutical composition of claim 1 further comprising a particle lubricant selected from the group consisting of talc, lactose, corn starch, ethyl cellulose, fatty acids or salts thereof, agar pectin, gelatin and acacia.

13. A method for administering a biologically active compound to an animal, the method comprising orally administering a pharmaceutical composition according to claim 1 to the animal.

14. A proliposomal composition prepared by lyophilization, spray drying in the presence or absence of a surfactant, or pan drying.
